(19) United States
(12) Patent Application Publication
(10) Pub. No.: US 2024/0325603 A1
(43) Pub. Date: Oct. 3, 2024

(54) PANCREATIC CELL RECEIVING MATRIX AND IMPROVED ARTIFICIAL PANCREAS DEVICE

(71) Applicants: UNIVERSITE GRENOBLE ALPES, Saint Martin D'Heres (FR); CENTRE HOSPITALIER UNIVERSITAIRE GRENOBLE, La Tronche (FR); INSTITUT POLYTECHNIQUE DE GRENOBLE, Grenoble (FR); CENTRE NATIONAL DE LA RECHERCHE SCIENTIFIQUE, Paris (FR)

(72) Inventors: Abdelkader ZEBDA, Grenoble (FR); Philippe CINQUIN, Saint Nazaire Les Eymes (FR); Awatef BEN TAHAR, Grenoble (FR); Emilie TUBBS, Vaulnaveys-Le-Haut (FR); Pierre-Yves BENHAMOU, Meylan (FR); Sandrine LABLANCHE, Bernin (FR)

(21) Appl. No.: 18/575,587

(22) PCT Filed: Jun. 29, 2022
(86) PCT No.: PCT/EP2022/067873

§ 371 Date: Dec. 29, 2023

(30) Foreign Application Priority Data
Jun. 30, 2021 (FR) 2107086

(51) Int. Cl. A61L 27/38; A61F 2/02; A61L 27/16; A61L 27/20; A61L 27/56

(52) U.S. Cl. CPC A61L 27/3804; A61F 2/022; A61L 27/16; A61L 27/20; A61L 27/56; A61L 2430/34

(57) ABSTRACT

A pancreatic cell receiving matrix including a semi-permeable wall and a porous body including a first set of cavities including pancreatic cells, and a second set of cavities free of pancreatic cells. The first set of cavities and the second set of cavities are not fluidically connected to one another. The cavity or cavities left free of cells create paths for the nutrients and gases to diffuse in the matrix, and they form a nutrient and gas reserve area in the matrix. The matrix limits and preferably prevents nutrient and gas depletion of the pancreatic cells. Their mortality is therefore reduced, allowing better insulin delivery.

### TECHNICAL FIELD OF THE INVENTION

The present invention relates to the field of implantable artificial pancreas devices. A particularly advantageous use thereof is the production of insulin for the treatment of diabetes, and in particular type 1 diabetes.

### PRIOR ART

Diabetes is a disease with a worldwide range, which affects both children and adults and which requires lifelong heavy therapeutic care. In 2016, 8% of the French population was affected by this disease. On the worldwide scale, the number of diabetic persons is estimated at 422 million.

Diabetics must constantly monitor their blood glucose level (commonly abbreviated BGL, can also be called glycemia) in order to receive a treatment in case of hypoglycemia (abnormally low BGL) or hyperglycemia (abnormally high BGL).

Numerous complications can be avoided with an anticipation of these critical glucose levels. Indeed, diabetes can cause disorders of the heart, the kidneys, the retina or the nervous system in the case of a strong increase or decrease in glycemia. Moreover, it should be noted that the costs of the treatments of these complications represent the majority of the spending related to therapy for diabetes.

It is therefore crucial, from a health and economic point of view, to find a means to maintain glycemia at a normal level in diabetic individuals. At present, there are methods for verification of glycemia by the patient. Among these, the BGL can be measured sporadically by an electrochemical device using enzymatic electrodes printed on strips, quantifying glycemia from a drop of blood. These devices, commonly called point of care devices, allow diabetics to independently monitor their glycemia. The patient is, however, lead to prick themselves several times per day to check their glycemia and thus avoid any complications from the diabetes. This monitoring is restrictive for the patient and often leads to losses of sensitivity in the zones of the body frequently pricked.

Alternatively, the transplantation of pancreatic islets is a proposed treatment for type 1 diabetes, in particular for diabetic patients for whom the checking and the monitoring of glycemia are difficult to implement and cause complications.

To carry out this transplantation of pancreatic islets, also called islet allo-transplantation, doctors generally collect islets comprising healthy beta pancreatic cells coming from the pancreas of a deceased organ donor. The doctors then inject the healthy islet cells into the patient via a vein transporting the blood towards the liver. Once fastened onto the liver of the patient, these islets start to produce and release insulin into the body of the patient. Several injections of grafted islet cells are often necessary to stop the use of insulin.

However, after explanation, the vascularization around the transplanted islets requires a certain time during which a lot of islets are degraded by lack of oxygen. The mortality of the islets is estimated at approximately 30% to 40% of the number of islets transplanted. Moreover, a systemic immunosuppression must be carried out in the patient to avoid a rejection of the transplanted islets.

Several approaches have been proposed to limit these problems related to the lack of oxygen. Certain approaches comprise molecular and pharmacological treatments, to protect the pancreatic islets against hypoxic stress and immune reactions. These treatments remain, however, cumbersome for the patient and of limited effectiveness.

Other solutions involve encapsulating the islets in polymer matrices insulating them from the inner environment of the body. The islets are thus protected from the attacks of the immune systems. On the one hand, there are approaches of microencapsulation of the islets. Because of their size, the microcapsules remain, however, difficult to recover to renew the islets and thus to ensure a delivery of insulin over the long term.

On the other hand, there are approaches of macroencapsulation of the islets. These approaches involve in general mixing the islets with a polymer that is bulk hardened. Because of their size, the diffusion of the nutrients and of the oxygen in the macrocapsules is limited however, in particular in the core of the macrocapsule. Moreover, surface biofouling leads to an insulation of the macrocapsule, further limiting the diffusion of the nutrients and of the oxygen. The mortality of the islets remains too high to allow good delivery of insulin.

Furthermore, devices of the artificial pancreas type are known. The document U.S. Pat. No. 5,425,764 A1 describes an implantable artificial pancreas comprising a chamber containing the islets of Langerhans provided with inlet and outlet channels to supply the islets, an open vascularization chamber, filled with a foam and with a semi-permeable membrane separating the chambers. This document attempts to solve the problem of improving the service life of the islets by protecting them, in a chamber, from inflammatory agents for example. However, the vascularization near the chamber, even though it is aimed at a supply of oxygen, also brings molecules involved in inflammatory reactions and the appearance of biofouling. The document US 2018/0263238 also describes a device for encapsulating cells producing insulin comprising layers formed by a first membrane and by a second membrane, welded to each other to form channels. Certain channels receive islets and certain channels are devoid of islets to form fluid transport channels allowing to bring nutrients to the islets. This device, although it attempts to solve the issue of bringing nutrients to the islets, does not allow good effectiveness in that the nutrients are brought in channels neighboring the channels containing the islets, but diffuse into the outside environment which is voluntarily open to vascularization. Thus, the supply of the nutrients to the islets passes first through this medium which depletes the quantity of nutrients that can diffuse once again towards the islets contained in other channels.

One object of the present invention is therefore to propose a solution allowing a delivery of insulin that is improved with respect to the existing solutions.

The other objects, features and advantages of the present invention will appear upon examining the following description and the appended drawings. It is understood that other advantages can be incorporated.

### SUMMARY OF THE INVENTION

To reach this goal, according to one embodiment a matrix for receiving pancreatic cells is provided comprising:
a semi-permeable wall at least partly defining an inner volume,
a porous body, preferably made from at least one polymer, disposed in the inner volume, comprising:
   a first cavity set comprising pancreatic cells,
   a second cavity set devoid of pancreatic cells,
   the first cavity set and the second cavity set not being fluidly connected to one another.

The arrangement of the pancreatic cells in the cavity or cavities of the porous body allows to control the distribution of the cells in the porous body, by opposition to the solutions of macroencapsulation in which the cells are set in a polymer matrix that is bulk hardened. Thus, the diffusion of the nutrients and of the gases to each cavity comprising cells can be improved. The cavity or cavities left free of cells further create paths for diffusion of the nutrients and of the gases in the matrix, and form one or more nutrient and gas reserve zones in the matrix. By synergy, the matrix limits and preferably avoids the depletion of nutrient and of gas in the pancreatic cells. Their mortality is thus reduced, allowing a better delivery of insulin.

A second aspect of the invention relates to an artificial pancreas device intended to be implanted in the human or animal body and comprising the matrix for receiving pancreatic cells according to the first aspect.

A third aspect of the invention relates to a method for preparing the matrix for receiving pancreatic cells comprising:
providing a matrix capable of forming the reception matrix according to the first aspect,
a seeding of the first cavity set of said matrix by pancreatic cells.

The matrix capable of forming the reception matrix can comprise at least the porous body comprising:
a first cavity set intended to comprise pancreatic cells, and more particularly devoid of pancreatic cells,
a second cavity set devoid of pancreatic cells,
the first cavity set and the second cavity set not being fluidly connected to one another devoid of pancreatic cells.

The semipermeable wall can for example be added after the seeding of the pancreatic cells. The matrix capable of forming the reception matrix can be the reception matrix according to the first aspect, devoid of pancreatic cells. More particularly the first cavity set can be devoid of pancreatic cells.

A third aspect of the invention relates to a method for delivering insulin comprising the implantation of the artificial pancreas device according to the second aspect, in a human or animal body.

According to another aspect, the invention relates to a reception matrix capable of receiving pancreatic cells comprising:
a semipermeable wall at least partly defining an inner volume,
a porous body, preferably made from at least one polymer, disposed in the inner volume, comprising:
   a first cavity set intended to receive pancreatic cells,
   a second cavity set not intended to receive pancreatic cells,
   the first cavity set and the second cavity set not being fluidly connected to one another.

### BRIEF DESCRIPTION OF THE DRAWINGS

The goals, objects, as well as the features and advantages of the invention will be clearer from the detailed description of an embodiment of the latter that is illustrated by the appended drawings in which:

FIGS. 1 and 2 show a transverse cross-sectional view of the matrix according to two exemplary embodiments.

FIGS. 3 to 5 show a longitudinal cross-sectional view of a device comprising the matrix according to three exemplary embodiments.

FIGS. 6A to 6C show a detail view of a device comprising the fluidic module illustrated in FIG. 5, according to three exemplary embodiments.

FIG. 7 shows a transverse cross-sectional view of a device comprising the matrix according to another exemplary embodiment of the fluidic module.

FIG. 8 shows a transverse cross-sectional view of a device comprising the matrix and electrodes for the electrolysis of the bodily fluid, according to an exemplary embodiment.

FIGS. 9A and 9B show two views of a device comprising the matrix and electrodes for the electrolysis of the bodily fluid, according to another exemplary embodiment.

The drawings are given as examples and are not limiting to the invention. They constitute schematic representations of a principle intended to facilitate the comprehension of the invention and are not necessarily on the scale of the practical uses. In particular, the relative dimensions are not representative of reality.

### DETAILED DESCRIPTION OF THE INVENTION

Before undertaking a detailed review of embodiments of the invention, optional features that can optionally be used in association or alternatively are stated below.

According to one example, the first cavity set comprises at least one cavity, and preferably several cavities. These cavities can be interconnected or in an equivalent manner fluidly connected to each other or not.

According to one example, the second cavity set comprises at least one cavity, and preferably several cavities. These cavities can be interconnected or in an equivalent manner fluidly connected to each other or not.

According to one example, for two consecutive cavities of the first set taken according to a first direction, and preferably for each two consecutive cavities of the first set taken according to the first direction, a cavity of the second set is disposed between the two consecutive cavities of the first set. The cavities comprising cells are thus alternated according to the first direction with cavities devoid of cells, to distribute the reserve zones in the matrix. The first direction is preferably perpendicular to the direction of principal extension of said consecutive cavities of the first set.

According to one example, for two consecutive cavities of the first set taken according to a second direction, and preferably for each two consecutive cavities of the first set taken according to a second direction, a cavity of the second set is disposed between the two consecutive cavities of the first set. The cavities comprising cells are thus alternated with cavities devoid of cells according to two directions. The cavities comprising cells can thus be surrounded by zones of reserves. The distribution of these reserve zones is thus even further improved, to facilitate the access of the cells to the nutrients and to the gases. The second direction is preferably perpendicular to the direction of principal extension of said consecutive cavities of the first set. Preferably, the first direction is substantially perpendicular to the second direction.

According to one example, a distance D1 between at least one cavity of the second set and at least one directly neighboring cavity of the first set, preferably each directly neighboring cavity of the first set, taken from the center of the cavity of the first set to the center of the at least one cavity of the second set, is substantially greater than or equal to 0.5 mm (10⁻³ m), preferably 1.5 mm. The distance D1 can be substantially less than or equal to 3 cm (10⁻² m). The distance D1 can be substantially between 0.5 mm and 3 cm.

According to one example, the distance D1 between each cavity of the second set and at least one directly neighboring cavity of the first set, preferably each directly neighboring cavity of the first set, taken from the center of the cavity of the first set to the center of the at least one cavity of the second set, is substantially greater than or equal to 0.5 mm (10⁻³ m), preferably 1.5 mm. The distance D1 can be substantially less than or equal to 3 cm (10⁻² m). The distance D1 can be substantially between 0.5 mm and 3 cm.

According to one example, the first set comprises a plurality of cavities interconnected and fluidly connected to each other. According to one example, the first set comprises a plurality of cavities interconnected and fluidly connected, preferably directly, to an injection channel. The cavities of the first set are for example interconnected by being fluidly connected, preferably directly, to the injection channel. The delivery of insulin is improved over the long term by allowing a renewal of the cells.

According to one example, the second set comprises a plurality of interconnected cavities. According to one example, the second set comprises a plurality of cavities interconnected and fluidly connected to each other.

According to one example, each cavity of the first set has a transverse cross-section having a smallest dimension, for example its diameter, substantially greater than the size of the pancreatic cells or of the islets of pancreatic cells. In a manner synergetic with the interconnection of the cavities of the first set, the cells are free to circulate in the cavities of the first set, which facilitates the renewal of the cells of the matrix.

According to one example, the cavities of the first set extend in a direction of principal extension, for example in a manner parallel to each other.

According to one example, the cavities of the second set extend in a direction of principal extension, for example in a manner parallel to each other.

According to one example, the cavities of the first and of the second set extend in a direction of principal extension, for example in a manner parallel to each other.

According to one example, the cavities of the first and of the second set extend according to their direction of principal extension over a length substantially greater than or equal to 70%, preferably substantially greater than or equal to 80%, of the length of the porous body taken according to the same direction.

According to one example, the porous body is made from or made of chitosan, polyacrylamide, poly(p-phenyl-p-phthalamide), aramid nanofibers, polyvinyl alcohol.

According to one example, the porous body has a cutoff threshold between 5.8 kDa and 8 kDa.

According to one example, the semipermeable wall has a cutoff threshold substantially between 5.8 kDa and 8 kDa.

According to one example, the pancreatic cells are comprised in islets of pancreatic cells. Preferably the islets are microencapsulated.

According to one example, the device further comprises a fluidic module configured to create a flow of liquid around and/or inside the matrix.

According to one example, the fluidic module comprises a coating of the matrix made from an electroactive polymer, the coating being capable of deforming the matrix. According to one example, the coating is additional to the wall of the matrix. According to one example the coating has a cutoff threshold substantially greater than or equal to 5.8 kDa, for example substantially between 5.8 kDa and 8 kDa.

According to one example, the fluidic module comprises a pump, the pump being configured to induce a flow of liquid around and/or inside the matrix.

According to one example, the fluidic module is fluidly connected to the second cavity set, preferably the second set comprising a plurality of cavities. According to one example, the cavities of the second set are interconnected, for example by a channel.

According to one example, the fluidic module comprises a tank and a pump, the pump being configured to induce a flow of liquid from the tank to inside the cavities of the second set.

According to one example, the tank comprises at least one out of a nutrient for the pancreatic cells and an anti-inflammatory compound.

According to one example, the fluidic module comprises a pump configured to induce a flow of liquid from a liquid outside of the device, for example a bodily fluid, to inside the cavities of the second set.

According to one example, the fluidic module comprises a regulator configured to regulate at least one parameter of the formation of the flow by the fluidic module.

According to one example, the device further comprises at least one anode and at least one cathode, and a source of electric energy. The anode and the cathode can be electrically connected to the source of electric energy, so that, in the presence of bodily fluid, a closed electric circuit is formed so as to produce dihydrogen at the cathode and dioxygen at the anode, by electrolysis of the bodily fluid. The pancreatic cells are thus preserved by a production of dioxygen and of dihydrogen by electrolysis, to improve the release of insulin in the inner environment.

According to one example, the device is configured to electrolyze the bodily fluid only in liquid form. The electrolyzed bodily fluid does not therefore comprise a gaseous fraction.

According to one example, at least one out of the cathode and the anode is disposed in the inner volume at least partly defined by the semipermeable wall of the matrix. The device is thus simplified with respect to the existing solutions that provide two distinct devices or compartments for the electrolysis and the cells between which the electrolysis gases are transported.

According to one example, at least one out of the cathode and the anode is disposed in the porous body of the matrix. According to one example, the cathode and the anode are disposed in the porous body of the matrix.

According to one example, at least one out of the cathode and the anode is disposed on, preferably directly on, the porous body of the matrix.

According to one example, the cathode and the anode are disposed in contact, preferably directly in contact, with the outer contour of the body of the matrix.

In the rest of the description, the term “on” or “in contact” does not necessarily mean “directly on” or “directly in contact”. Thus, when it is indicated that a part or that a member A1 bears “on” a part or a member B1, this does not mean that the parts or members A1 and B1 are necessarily in direct contact with one another. These parts or members A1 and B1 can either be in direct contact or bear one on another via one or more other parts.

In the detailed description that follows, terms such as “longitudinal”, “transverse”, “upper”, “lower”, “inner”, “outer” can be used. These terms must be interpreted in a relative manner in relation to the normal position of use of the reception matrix and/or of the artificial pancreas device. For example, the notion of “inner” corresponds to the faces or elements facing towards the inside of the matrix and/or of the device. The notion of “outer” corresponds to the faces or elements facing towards the outside of the matrix and/or of the device. For example, the cavities extending according to a direction of principal extension, “longitudinal” means parallel to this direction, and “transverse” means perpendicular to this direction.

The expression “A fluidly connected to B” is synonymous with “A is in fluid connection with B” and does not necessarily mean that there is no member between A and B. Thus, these expressions mean a fluid connection between two elements, wherein this connection can be direct or not. This means that it is possible that between a first element and a second element that are fluidly connected, there is a path of a fluid via one or more ducts or cavities or channels, optionally an additional member, this path being distinct from the simple diffusion of the fluid through the material of the porous body, wherein this path can comprise other elements or not.

On the contrary, the term “directly fluidly connected” means a direct fluid connection between two elements. This means that between a first element and a second element that are directly fluidly connected no other element is present, other than a duct/cavity/channel or several ducts/cavities/channels.

A parameter “substantially equal to/greater than/less than” a given value means that this parameter is equal to/greater than/less than the given value, plus or minus 10%, or even plus or minus 5%, of this value.

An element “made from” a material means an element comprising this material and optionally other materials. “Made from” a material is meant as the fact that said material forms a majority relative to the possible other materials.

Porosity of an element or of a material means the volume not occupied by the material forming it, relative to the apparent volume of the element or of the material. This volume proportion can be occupied by the surrounding environment of the element or of the material, emptiness, gas or a liquid, for example water. In the context of the present invention, the porosity of the material is meant in a manner distinct from the cavities of the first set and of the second set.

“Cutoff threshold” of a membrane, of a body or of a member means the molecular weight cutoff or the dimension threshold for which at least 90%, preferably at least 95%, preferably at least 99%, even more preferably 100%, of the species having a molecular weight or a dimension greater than or equal to the molecular weight cutoff or the dimension threshold are blocked by the membrane, the body or the member.

The matrix 1 for receiving pancreatic cells and the implantable artificial pancreas device 2 comprising it are now described according to several exemplary embodiments.

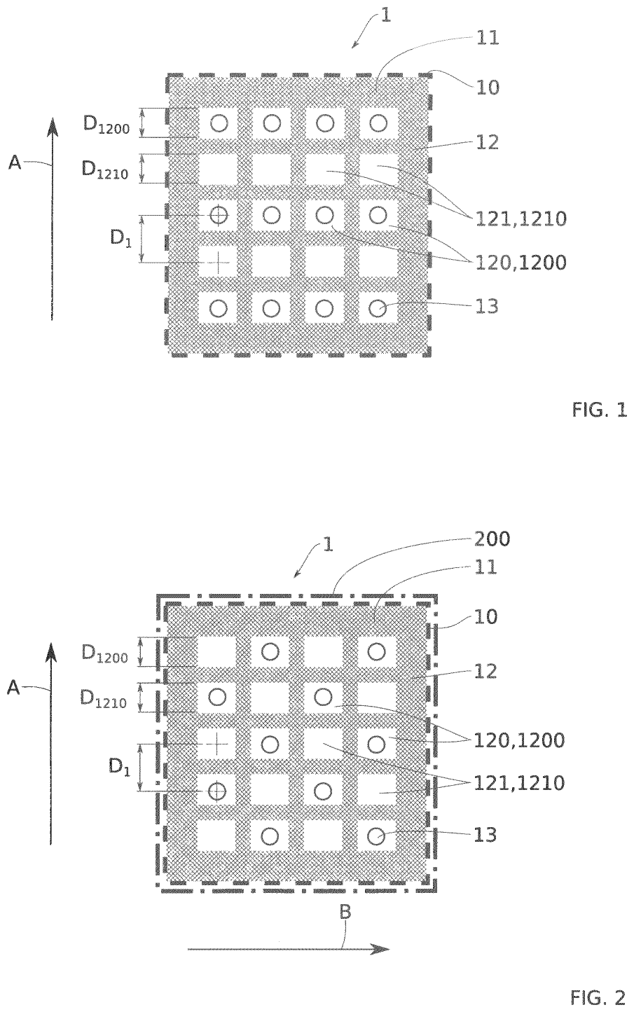

The artificial pancreas device 2 is intended to be implanted in the human or animal body so as to deliver insulin. For this, the device 2 comprises a matrix 1 for receiving pancreatic cells. The matrix 1 comprises, as illustrated by FIGS. 1 and 2, a semipermeable wall 10 at least partly defining an inner volume 11. A porous body 12 is disposed in the inner volume 11. The porous body 12 is configured to receive pancreatic cells 13, in order to deliver insulin. The pancreatic cells 13 can be isolated or grouped together in the form of pancreatic islets. A pancreatic islet comprises at least one pancreatic cell, and preferably a plurality of pancreatic cells. Hereinafter, it is considered in a non-limiting manner that the matrix 1 receives pancreatic islets 13, also designated as islets. The pancreatic cells 13 are for example Langerhans beta cells. The pancreatic cells 13 can be stem cells intended to become pancreatic cells or pancreatic cells coming from stem cells.

To receive the islets 13, the porous body 12 comprises a first set 120 of cavities 1200 and a second set 121 of cavities 1210. For this, the porous body 12 comprises walls defining the first cavity 1200 set 120 and the second cavity 1210 set 121. Advantageously, the walls are formed by the interface between the porous body and the cavities. Preferably, the walls are not formed by a layer of additional material. Cavity set means a cavity group, the group comprising at least one cavity. Hereinafter, it is considered in a non-limiting manner that each set 120, 121 comprises a plurality of cavities 1200, 1210. The cavities 1200 of the first 120 comprise islets 13, while the cavities 1210 of the second set 121 do not comprise any islets. The cavities 1200 and the cavities 1210 are not fluidly connected to each other, that is to say form two distinct fluidic assemblies. In an equivalent manner, the cavities 1200 do not fluidly communicate with the cavities 1210, except by the diffusion of a liquid through the walls of the porous body 12 and advantageously inside the porous body. The cavities 1210 create paths for the diffusion of the nutrients and of the gases in the matrix 1, and thus form reserve zones of nutrient and of gas to facilitate the supply of the islets 13 comprised in the cavities 1200.

As for example illustrated by FIGS. 1 and 2, in the transverse cross-section of the matrix 1, the porous body 12 can form a network of cavities, for example in the form of a mesh. According to one example, the cavities 1200 and 1210 alternate so that the cavities 1210 form zones of reserves between the cavities 1200 comprising the islets 13. As for example illustrated by FIG. 1, according to a first direction A, a cavity 1210 can be disposed between two consecutive cavities 1200 of the first set, preferably between each consecutive cavity 1200 pair of the first set. Consecutive means which follow each other directly when only considering the cavities 1200 of the first set 120. The zones of reserves are thus distributed in the porous body 12 according to the first direction A, between the cavities 1200 receiving the islets 13. The porous body 12, in its transverse cross-section, can comprise at least one row of cavities 1200 alternating with at least one cavity 1210 row.

As for example illustrated by FIG. 2, a cavity 1210 can be disposed between two consecutive cavities 1200 of the first set also according to a second direction B, preferably between each consecutive cavity 1200 pair of the first set. Consecutive means here again which follow each other directly when only considering the cavities 1200 of the first set 120. The zones of reserves are thus distributed in the porous body 12 according to the first direction A and the second direction B, to further improve the distribution of the reserve zones and thus the diffusion of the nutrients and of the gases to the islets 13 comprised in the cavities 1200. The porous body 12, in its transverse cross-section, can comprise an alternation of cavities 1200 and of cavities 1210 according to these two directions. According to the first direction A and/or the second direction B, and preferably both, among the cavities that surround it, a cavity 1200 of the first set 120 can be surrounded only by directly neighboring cavities 1210 of the second set 121, and inversely for a cavity 1210.

According to one example, the first direction A and the second direction B are substantially perpendicular. The first direction A and the second direction B can be orthogonal to the direction of principal extension of the cavities 1200, 1210. The first direction A and the second direction B can be parallel to, and preferably included in, the plane of a transverse cross-section of the matrix 1.

The distance between the cavities 1200, 1210 can further be configured so as to allow a good distribution of the zones of reserves and of the cavities 1200 receiving the islets 13 in the porous body 12. For this, the distance D1, for example according to the first direction A and/or the second direction B, between a cavity 1210 and at least one directly neighboring cavity 1200, and preferably each directly neighboring cavity 1200, can be substantially greater than or equal to 0.5 mm. This distance allows to improve the provision of the nutrients for the islets 13 from the cavity 1210 into the cavity 1200. This distance further allows a good mechanical strength of the matrix. The distance D1 is taken from the center of a cavity 1210 to the center of the other cavity 1210. According to one example, the cavities 1200 and the cavities 1210 are equidistant from each other. The distance D1 is for example taken in the plane of a transverse cross-section of the matrix 1.

According to one example, the distance D1 between a cavity 1210 and at least one directly neighboring cavity 1200, for example according to the first direction A and/or the second direction B, and preferably each directly neighboring cavity 1200, is substantially less than or equal to 3 cm, preferably 2 cm. Thus, the bulk associated with the matrix 1 can be reduced for the same number of cavities, thus reducing a possible discomfort in the patient in whom the device 2 is implanted.

The cavities 1200 of the first set 120 are now described. According to one example, each cavity 1200 has a transverse cross-section having a smallest dimension D1200 substantially greater than or equal to the size of the islets 13. According to one example, each cavity 1200 has a transverse cross-section having a smallest dimension D1200 substantially greater than or equal to the size of at least one islet 13. In an equivalent manner, the transverse cross-section of a cavity 1200 defines a shape, the smallest dimension D1200 of which is substantially greater than or equal to the size of the islets 13. Thus, the cells are not constrained and fastened by the cavities 1200, which facilitates the supply of the islets 13. The transverse cross-section of a cavity 1200 is more particularly taken substantially perpendicularly to its direction of principal extension.

According to one example, the cavities 1200 having a circular transverse cross-section, the dimension D1200 correspond to the diameter of the transverse cross-section. The cavities 1200 can have a transverse cross-section of any other shape, for example rectangular, square or ellipsoid.

For example, in humans an islet 13 has a size, for example a diameter, of 130 μm on average; in small animals such as mice, an islet 13 has a size, for example a diameter, of 50 μm on average. According to one example, D1200 is substantially greater than or equal to 100 μm, preferably 150 μm, preferably 200 μm, preferably 300 μm, and more preferably 400 μm. D1200 can be substantially less than or equal to 5 mm. Thus, the bulk associated with the matrix can be reduced for the same number of cavities, thus reducing a possible discomfort in the patient.

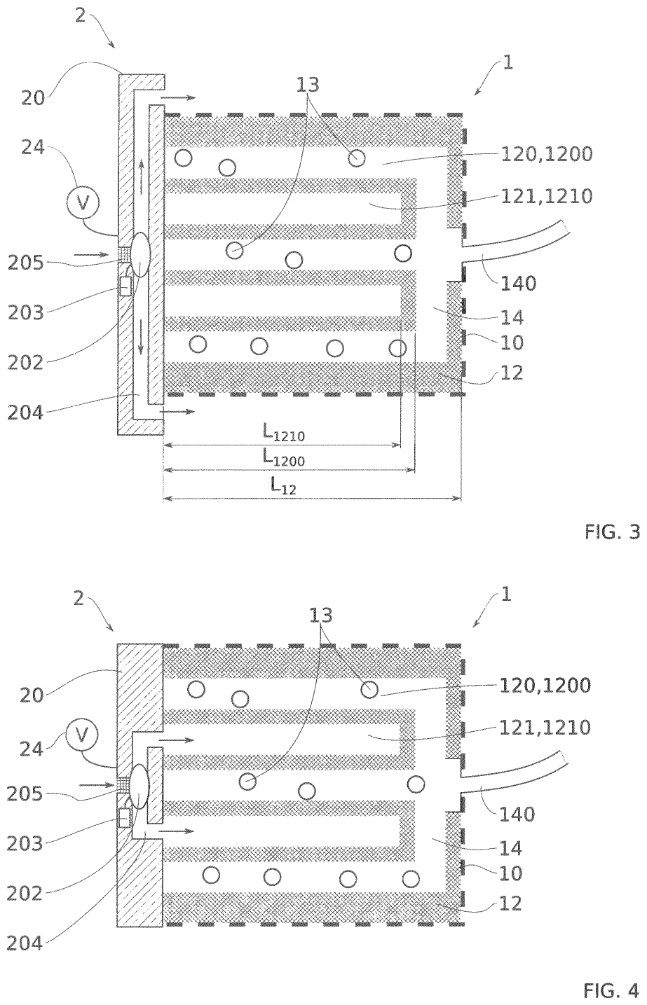

The cavities 1200 of the first set 120 can be fluidly not connected to each other. Preferably, the cavities 1200 of the first set 120 are interconnected with each other, that is to say fluidly connected to each other. The cavities 1200 of the first set 1200 can be interconnected so as to allow the circulation of the islets between each cavity 1200 of the first set 120. In a manner synergetic with the dimension D1200 of the cavities, the islets 13 are thus free to circulate between the cavities 1200 of the first set 120. A good distribution of the islets 13 can thus be obtained. As for example illustrated in FIG. 3, the cavities 1200 can be fluidly connected, preferably directly, to an injection channel 14. The islets 13 can thus be renewed via a simple suction and an injection of new islets into the injection channel 14, without requiring the removal or the replacement of the porous matrix 1, and thus without an operation. The device 2 comprising the matrix 1 can thus be implanted and deliver insulin over the long term. The delivery of insulin is consequently improved over the long term. According to one example, the injection channel 14 is configured so as to fluidly connect, preferably directly, an end of each cavity 1200. According to one example, the injection channel 14 extends substantially perpendicularly to the direction of principal extension of the cavities 1200.

According to one example, the injection channel 14 is fluidly connected, preferably directly, to an injection chamber 140. The islets can thus be injected from the injection chamber 140, into the cavities 1200. Preferably the injection chamber 140 is configured to have a loading end disposed outside of the body of the patient, in order to allow the recharge of the islets 13 by the injection chamber 140 from outside the body of the patient.

As for example illustrated by FIG. 3, the cavities 1200 can extend according to a main direction in a manner parallel to each other. The cavities 1210 can extend according to a main direction in a manner parallel to each other. The structure of the porous body is thus simplified, while being compatible with the alternations of the cavities 1200 and 1210 described above. These cavities can extend according to this direction, over a length L1200 and/or L1210 substantially greater than or equal to 70%, preferably substantially greater than or equal to 80%, of the length L12 of the porous body 12 taken according to this direction. Thus, a distribution of the islets 13 and/or of the zones of reserves is allowed over the length L12 of the porous body 12.

As for example illustrated by FIGS. 1 and 2, each cavity 1210 can have a transverse cross-section having a smallest dimension D1210 substantially equal to the dimension D1200 of the cavities 1200. It should be noted that it is possible for D1210 to be less than or greater than D1200. According to one example, the cavities 1210 having a circular transverse cross-section, the dimension D1210 corresponds to the diameter of the transverse cross-section. The cavities 1210 can have a transverse cross-section of any other shape, for example rectangular, square or ellipsoid.

The cavities 1210 of the second set 121 can be fluidly not connected to each other. Preferably, the cavities 1210 of the second set 121 are interconnected with each other, that is to say fluidly connected to each other. The cavities 1210 thus create interconnected paths for diffusion of the nutrients and the gases favoring a homogeneous distribution of the nutrients and of the gases in the porous body 12. As for example illustrated in FIG. 5, the cavities 1210 can be fluidly connected, preferably directly, to a channel 15. According to one example, the channel 15 is configured so as to fluidly connect, preferably directly, an end of each cavity 1210. According to one example, the channel 15 extends substantially perpendicularly to the direction of principal extension of the cavities 1210. The channel 15 and the injection channel 14 can be located with respect to one another opposite to the porous body 12 according to the direction of principal extension of the cavities 1200, 1210.

The porous body 12 can be made from or made of a material capable of letting the nutrients and the gases pass through, as well as the insulin produced by the islets, and of blocking the islets 13. For this, the material can have a cutoff threshold preferably substantially greater than or equal to the molecular weight of insulin, or approximately 5.8 kDa (1 Da being equivalent to 1 g/mol in the International System of Units). To block the islets 13, the material of the porous body 12 can have a cutoff threshold preferably substantially less than or equal to the size of the islets, for example their average diameter. According to one example, the material of the porous body can have a cutoff threshold substantially less than or equal to 100 μm, preferably less than 50 μm. In an equivalent manner, the material of the porous body 12 can have a porosity capable of allowing the passage of the nutrients and of the gases, as well as the insulin produced by the islets, and of blocking the islets 13. The porosity can be configured to let the passage of the molecules having a size at least equal to that of insulin be authorized. Since the nutrients and the gases have a molar or molecular weight of less than 5.8 kDa, their passage through the material of the porous body 12 is authorized. The porosity can be configured to block the passage of the elements having a size at least equal to that of the islets. The ranges of values presented above in relation to the cutoff threshold of the porous body 12 can be applied to the porosity of the porous body 12.

Advantageously, the porous body 12 has a cutoff threshold configured to limit or even prevent the passage of inflammatory agents. Advantageously, the porous body 12 has a cutoff threshold configured to limit or even prevent the vascularization of said porous body.

According to one example, the porous body 12 is made from a material capable of being 3D printed. According to one example, the porous body is made from a natural or synthetic polymer, preferably biocompatible and non-biodegradable. The material can be chosen from chitosan, for example cross-linked with genepine to become non-biodegradable in the physiological conditions, polyacrylamide (PAAm), poly(p-phenyl-p-phthalamide), aramid nanofibers, polyvinyl alcohol (PVA).

The semipermeable wall 10 can be made from or made of a material capable of letting the nutrients and the gases pass through, as well as the insulin produced by the islets, and of blocking the molecules of the immune system, for example the cytokines. For this, the wall 10 can have a cutoff threshold substantially between 5.8 kDa and 8 kDa. Via a cutoff threshold between 5.8 kDa and 8 kDa, the outer wall allows a communication of the bodily fluid between the inner environment of the body and the porous body 12 enclosing the islets 13. Thus, the nutrients necessary to the islets reach them from the inner environment, and the insulin produced by the islets 13 can be released into this environment for the regulation of glycemia. The passage into the matrix 1 of the molecules of the immune system, and in particular the cytokines, is blocked by the wall 11. Thus, the islets are protected from the reactions of the immune system. The systemic immunosuppression in the patient can thus be limited, and preferably be avoided. Advantageously, the semipermeable wall 10 is configured to reduce or even prevent the vascularization of the porous body. The matrix 1 and/or the device 2 can be configured so that the semipermeable wall 10 is directly in contact with the body of the patient.

According to one example, the semipermeable wall 10 is made from a natural or synthetic polymer, preferably biocompatible and non-biodegradable. Preferably the semipermeable wall 10 is made from a polymer having anti-biofouling properties. According to one example, the wall is made from or made of a hydrogel. According to an alternative or complementary example, the wall 10 is made from or made of at least one polymer out of polyethylene glycol (PEG), polyvinyl alcohol (PVA), a copolymer (ethylene vinyl alcohol) (EVOH), hexadimethrine bromide (more generally known under the brand name Polybrene) and carboxymethyl cellulose. The wall thus has a good biocompatibility and limits the phenomena of biofouling.

As stated above, the semipermeable wall 10 at least partly defines the inner volume 11. Indeed, the semipermeable wall 10 can entirely define the inner volume 11, as for example shown by FIGS. 1 and 2. Alternatively, the semipermeable wall 10 can only partly define the inner volume 11, as for example shown by FIGS. 3 to 5. The inner volume 11 can be defined by the semipermeable wall 10 and another element of the matrix 1 or of the device 2, for example the fluidic module 20 described in more detail later. The volume occupied by the islets 13 can be between 2.5 mL and 5 mL (10³ L). The volume occupied by the islets can represent substantially between 25% and 50% of the inner volume 11. The inner volume 11 can have dimensions representing a total volume substantially equal to or less than approximately 20 mL, preferably, in an increasing order of preference, 19 mL, 18 mL, 17 mL, 16 mL, 15 mL, 14 mL, 13 mL, 12 mL, 11 mL, 10 mL, 9 mL, 8 mL, 7 mL, 6 mL and 5 mL.

The matrix 1 is preferably configured in that the inner volume 11 is devoid of vascularization. The matrix 1 and more particularly the porous body 12 can have any shape, in particular parallelepipedic, cylindrical or in the shape of a disc. The porous body 12 can be in one piece or not. The porous body 12 can for example be formed by an assembly of porous and hollow fibers, the walls of the fibers defining the cavities 1200, 1210.

According to one example, the number of pancreatic cells 13 contained in the porous body 12 is between 10,000 and 50,000,000 cells/kg relative to the weight of the patient. Thus, the number of pancreatic cells is adapted to the insulin needs of the patient. The number of islets 13 contained in the porous body 12 can be substantially equal to 10,000 islets/kg relative to the weight of the patient. According to one example, considering an average weight of 70 kg of a typical patient, the number of pancreatic cells 13 contained in the porous body 12 is between 700,000 and 3,500,000,000 cells/kg, or approximately 700,000 islets for a typical patient of 70 kg.

As stated above, the pancreatic cells 13 can be comprised in islets of pancreatic cells. Preferably the islets are microencapsulated. Thus an additional protective barrier is disposed around the cells to increase their viability. Since the islets are contained in the matrix, the recovery of the microencapsulated islets is facilitated with respect to the solutions providing a dissemination of microencapsulated islets in the body of the patient.

The artificial pancreas device 2 is now described in reference to FIGS. 2 to 9B. The device can comprise a fluidic module 20 configured to create a flow of liquid around and/or inside the matrix 1. The biofouling is thus limited by the flow of liquid at the surface of the matrix and/or from the inside to the outside of the matrix. Moreover, the flow of liquid inside the matrix 1 allows to further improve the diffusion of the nutrients and of the gases in the porous body 12.

According to one example, the fluidic module 20 can be configured to exert a pressure on the matrix 1 so as to deform it sporadically, and for example at a certain frequency. The contractions of the matrix 1 thus induce fluidic shearing at the surface of the matrix 1, which limits the biofouling. For this, the fluidic module can comprise a coating 200 of the matrix 1. The coating 200 can only partly cover the matrix 1. The coating 200 can entirely cover the matrix 1. The coating 200 can be additional to the semipermeable wall 10, for example by being outside the wall 10 as for example illustrated by FIG. 2, or disposed between the wall 10 and the porous body 12. According to an alternative example, the coating 200 can be formed by the semipermeable wall 10 of the matrix 1. According to one example, the coating has a cutoff threshold substantially greater than or equal to 5.8 kDa, for example substantially between 5.8 kDa and 8 kDa, to let the insulin and the nutrients pass through and block the molecules of the immune system, for example the cytokines.

The coating 200 can be made from or made of an electroactive polymer, connected to a source of energy (not shown in FIG. 2). The electroactive polymer can for example be chosen from: the copolymer (ethylene-vinyl acetate) (EVAc), polyethylene (PE), polyaniline (PAni), for example nigraniline (NA) and leucoemeraldine (LM). This solution can however cause shearing forces that can affect the surrounding organs in the patient. The amplitude of the induced contractions of the matrix 1 can be chosen to minimize this effect.

According to an alternative or complementary example, the fluidic module 20 can be configured to induce a flow of liquid around the matrix 1, and more particularly at the surface of the matrix 1, as for example illustrated by FIG. 3. The fluidic module 20 can for this comprise a pump 202 configured to suck up liquid and inject the suctioned liquid at the surface of the matrix 1, for example by ducts 204 opening at the surface of the matrix 1.

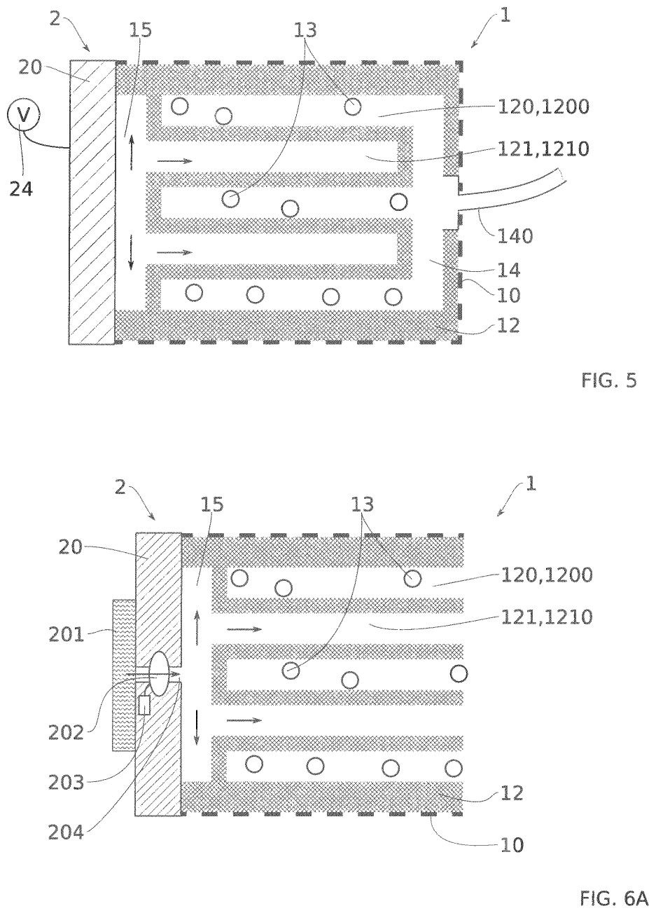

According to an alternative or complementary example, the fluidic module 20 can be fluidly connected to the second cavity set 121, the second set 121 comprising at least one and preferably several cavities 1210. As for example illustrated by FIG. 4, the fluidic module 20 can be fluidly connected to the cavity or cavities 1210, preferably directly. The cavities 1210 can not be interconnected, as illustrated for example by FIG. 4. The cavities 1210 can be interconnected. As for example illustrated by FIGS. 5, 6A to 6C, the fluidic module 20 can be fluidly connected, preferably directly, to a channel 15 connecting the cavities 1210. The fluidic module 20 can be configured to induce a flow of liquid in the cavities 1210. The fluidic module 20 can for this comprise a pump 202 configured to suck up liquid and inject the suctioned liquid into the cavities 1210, for example in each of the cavities 1210 separately or via the channel 15, for example by one or more ducts 204 opening into each cavity 1210, or into the channel 15. As for example illustrated in FIG. 6C, the pump 202 can be disposed in the channel 15.

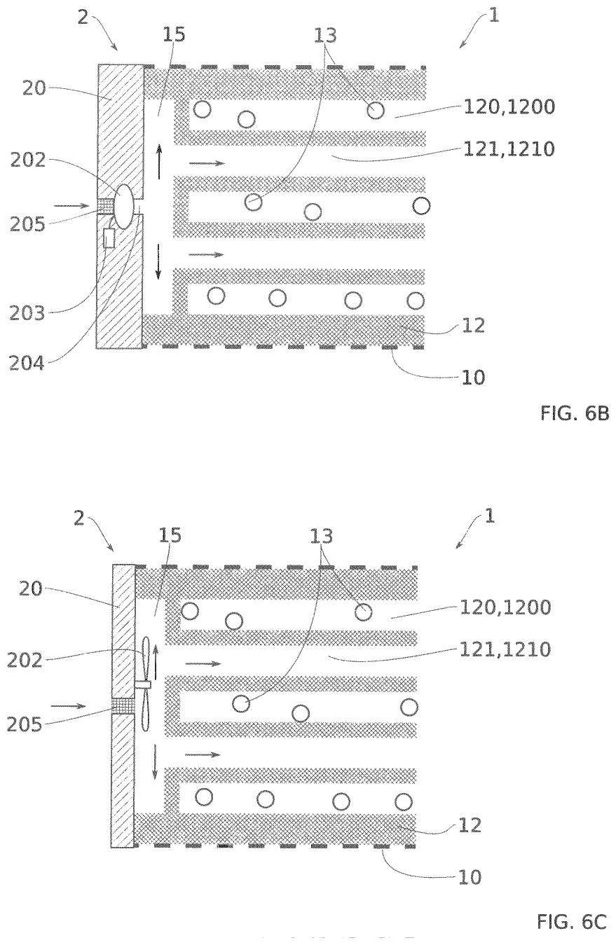

According to an alternative or complementary example, the fluidic module 20 can be configured to generate a flow transverse to the direction of principal extension of the cavities 1210, as for example illustrated by FIG. 7. The flow can propagate through the material of the porous body 12.

The fluidic module 20 can be configured to suck up bodily fluid outside of the device 2. Preferably, the fluidic module 20 comprises a membrane 205 configured to block the molecules of the bodily fluid that can foul the device 2, for example the fluidic module 20 and more particularly the pump 202, as for example illustrated by FIGS. 3, 4, 6B and 6C. The membrane 205 can for this have a cutoff threshold between 20 and 300 Da. The membrane can be made from or made of PVA, EVOH or chitosan.

The fluidic module 20 can be configured to suck up liquid coming from a tank 201, and cause a flow of liquid from the tank 201 into the cavities 1210 and/or at the surface of the matrix 1. It should be noted that the fluidic module 20 illustrated in FIG. 3 can comprise the tank 201, and the pump 202 can be configured to induce a flow of liquid from the tank 201 to the surface of the matrix 1. As for example illustrated by FIG. 6A, the pump 202 can be configured to induce a flow of liquid from the tank 201 into the cavities 1210, whether or not they are interconnected.

According to one example, the tank 201 comprises at least one out of a nutrient of the islets 13 and an anti-inflammatory compound. The fluidic module 20 can be configured so that the nutrients contained in the tank 201 are injected into the porous body 12, and more particularly into the cavities 1210, to provide the zones of reserves and facilitate the supply of the islets 13. The anti-inflammatory compound(s) allow a local anti-inflammatory treatment at the device 2. The fluidic module 20 can be configured so that the anti-inflammatory compound contained in the tank 201 is injected at the surface of the matrix 1. This allows to further limit the quantity of anti-inflammatory compound with respect to a systemic treatment.

According to one example, the tank 201 is configured to be rechargeable, for example via an injection. The tank 201 can for example be connected to an injection chamber or be rechargeable via an injection made into the tank. The contents of the tank 201 can thus be renewed.

Several types of pumps 202 are possible. As non-limiting examples, it is possible for the pump to be an acoustic pump, or a mechanical pump, for example a propeller or a mechanical valve. The pump 202 can be configured to generate a turbulent flow of liquid, in order for example to increase the shearing forces at the surface, and/or better favor the diffusion of the nutrients and of the gases to supply the islets 13. The pump 202 can be configured to generate an alternating flow of liquid, that is to say to inject liquid in a first direction according to a first configuration, and in a direction opposite to the first direction according to a second configuration. This is particularly advantageous with the use of the membrane 205, to strip the molecules that can accumulate on the membrane 205 and hamper the suction of the liquid by the pump 202.

The fluidic module 20 can further comprise a regulator 203 configured to regulate at least one parameter of the formation of the flow by the fluidic module 20. The regulator 203 thus allows to adjust the flow formed by the fluidic module 20 according to the needs, for example the speed of the fluid and/or the phases of actuation of the fluidic module 20. The regulator 203 can for example be configured to actuate the fluidic module 20, and in particular the pump 202, intermittently, between phases of generation of flow and phases of non-generation of flow by the fluidic module 20. The regulator 203 can for example be configured to modulate the speed of the flow generated by the fluidic module 20, for example the speed of the pump 202, so as to vary the intensity of the flow of liquid formed. This is particularly advantageous for modulating the flow according to the time following the implantation of the device 2. Typically, the flow of liquid can be more intense and/or generated more frequently in the first weeks after the implantation, then it can be slowed down or its frequency can be reduced.

The fluidic module 20, and more particularly the pump 202 and if necessary the regulator 203, can be electrically powered by a source of electric energy 24. The sources of energy 24 that are possible as an example are described later.

The device 2 can comprise electrodes: at least one anode 21 and at least one cathode 22, and a source of electric energy 23. The anode 21 and the cathode 22 can be electrically connected to the source of electric energy 23, so that, in the presence of bodily fluid, a closed electric circuit is formed so as to induce the electrolysis of the water of the liquid contained in the device 2 or in its environment, for example the bodily fluid. Dihydrogen at the cathode 2 and dioxygen at the anode 21 can be produced.

An oxygenation of the islets is thus carried out, coupled with the anti-inflammatory properties of the hydrogen allowing to reduce the immune reactions, and in particular the post-transplantation inflammatory reactions. The hydrogen is released into the inner environment by passing through the semipermeable wall of the matrix 1, to limit the inflammatory reactions that can occur around the device 2. The artificial pancreas device 2 allows to take advantage of the electrolyzer and artificial pancreas functions, while remaining of a simple structure.

Preferably, at least one out of the cathode 22 and the anode 21 is disposed in the inner volume 11, and preferably both electrodes 21, 22 are disposed in the inner volume 11. The electrolysis and the production of insulin are both carried out in the inner volume of the matrix, thus simplifying the device with respect to the solutions providing two distinct devices or compartments for the electrolysis and the cells between which the electrolysis gases are transported. The device 2 is also more compact and thus less invasive for the patient.

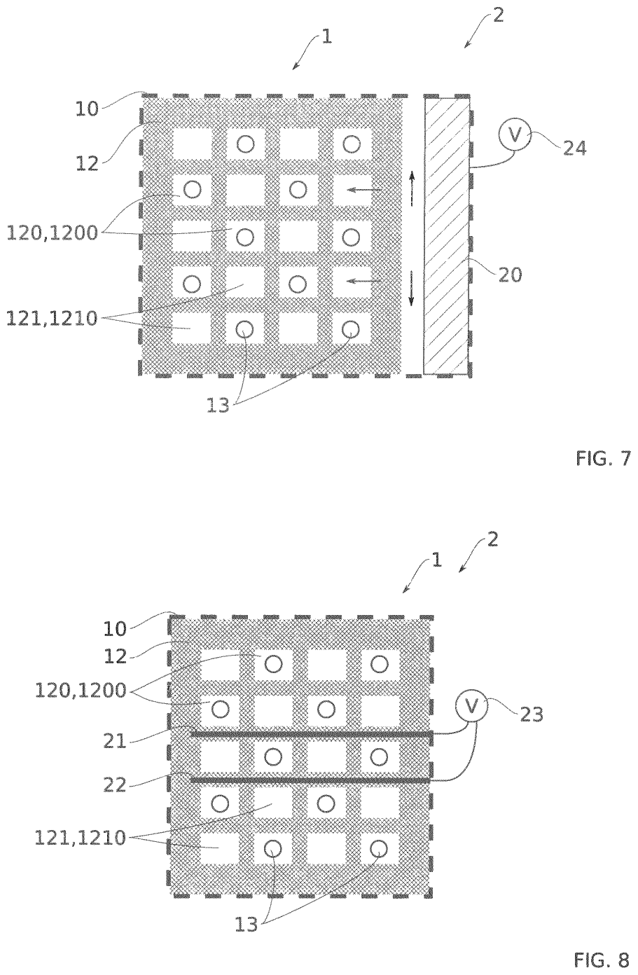

According to one example, the cathode 22 and/or the anode 21 are disposed in the porous body 12 of the matrix 1, for example between the cavities 1200, 1210. More particularly, the cathode 22 and/or the anode 21 can be disposed in the material of the porous body 12. The cathode and/or the anode 21 can be disposed in the walls of the porous body 12 defining the cavities 1200, 1210, as for example illustrated by FIG. 8. The production of the electrolysis gases is thus carried out near the cavities 1200 housing the islets 13.

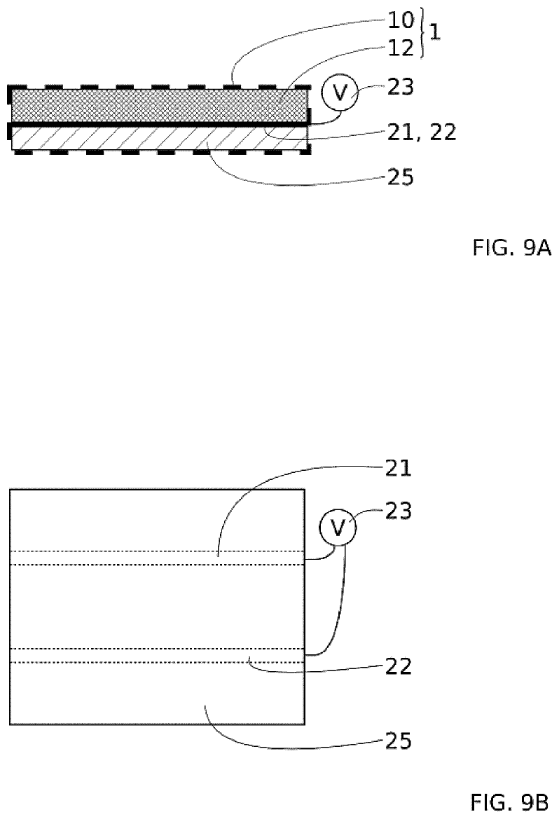

The cathode 22 and/or the anode 21 can alternatively or in addition be disposed on, preferably directly on, the porous body 12 of the matrix 1. The cathode 22 and/or the anode 21 can be disposed on, preferably directly on, the outer contour of the porous body 12, as for example illustrated by FIGS. 9A and 9B. The cathode 22 and/or the anode 21 can be disposed on a solid support 25. According to the relative arrangement of the electrodes with respect to the porous body 12, the delivery of a certain gas, in particular oxygen, to the islets, or to the surrounding environment, can be favored, in particular for hydrogen.

The electrodes 21, 22 are distant from one another so as to allow the operation of the device 2. The distance between the electrodes is defined by the value of the current of the electrolysis of the water. Indeed, for example at the cathode, the reduction of the protons creates a depletion layer, the thickness of which depends on the value of the reduction current. In all cases, the distance between electrodes is preferably greater than the thickness of the depletion layer. Generally, the distance separating them can be between approximately 0.1 mm and approximately 1 cm, in particular between approximately 0.2 and approximately 7 mm, preferably between approximately 0.5 and approximately 5 mm. This separation distance can be applied between two bulk (3D) electrodes disposed in parallel, or between two electrodes in two dimensions (2D) supported by two parallel supports or this can be the separation distance between two 2D electrodes disposed on the same support.

The electrodes can have a 2D geometry. These 2D electrodes can be manufactured by deposition of the material of the electrode on a support or two supports. A single support can be used for both electrodes under the condition that the support is not electrically conductive. As a support, mention can be made of a fine sheet of graphite, of platinum or of gold, a fine sheet allowing the diffusion of the gases called “gas diffusion layer”, a sheet of paper, of glass, of silicon. The deposition can be: physical deposition (for example PVD for physical vapor deposition, cathodic evaporation, lithography, plasma deposition), electrochemical, printing, spray, or mechanical compression, chemical deposition (for example CVD for chemical vapor deposition, sol-gel).

The electrodes can have a 3D geometry. They can be formed conventionally, preferably by compression, stereolithography, or 3D printing.

The composition of the electrodes is adapted to the function of each of them. They can be of the same material or of two different materials. They can be made from or made of carbon. Preferably, the type of carbon material is chosen from graphite, carbon nanotubes, graphene, activated charcoal or diamond. The material of the electrodes can be doped, in particular with platinum, with iron or with gold. The electrodes can be made from or made of platinum, gold, indium tin oxide (commonly abbreviated ITO), iridium or doped diamond, in particular at least the anode can be made of gold, or doped with gold. The electrodes can have a thickness substantially between 100 μm and 2 mm, according to a direction perpendicular to the face 101 intended to be in contact with the skin, in particular when the electrodes are in the form of studs, a bar or a sheet.

The electrodes, in particular when they are made of metal, for example made of gold or platinum, can also be blades, for example several centimeters long, several millimeters wide, several tens or hundreds of microns thick, or bare wires (category of the 3D shapes).

The electrodes can further each be encapsulated by a semipermeable membrane surrounding each electrode, not shown in the drawings, for example of the polyethersulfone, polyamide, poly(methyl methacrylate) (PMMA), chitosan, PVA type. The semipermeable membrane surrounding the anode 21 and/or the cathode 22 can be made from, and preferably be made of, fluoropolymer made from sulfonated tetrafluoroethylene, better known under the name Nafion®. This semipermeable membrane can have a cutoff threshold of less than 50 Da and surround the electrodes 21, 22. The membrane is configured to prevent the passage of the components of the bodily fluid to the electrodes, for example of the nutrients essential to the islets. Only the conductive ions, the molecules of water, as well as the gases produced, can circulate through this membrane to produce per electrode the dioxygen and the dihydrogen. The electrochemical reactions of the components of the bodily fluid at the electrodes are thus limited, and preferably prevented. Moreover, the phenomena of adsorption of these components at the surface of the electrodes are prevented, thus avoiding possible parasitic reactions. The islets 13 are thus protected from these reaction products that can be harmful to them.

The sources of energy 23, 24 powering the device 2 are now described. These sources can be distinct or the same. Hereinafter, it is considered in a non-limiting way that the sources of energy 23, 24 are the same, that is to say that one source of energy powers the various elements requiring an electric power supply. The source of energy 23, 24 can be integrated into the device. Alternatively, the source of energy 23, 24 can be remote from the device 2 and connected to the device 2 by electric connections. Since the source of electric energy generally represents a significant volume of this type of device, by being remote it can be implanted at a location distinct from the site of implantation of the device 2, and thus minimize the discomfort caused in the patient. The source of energy can be configured to be outside of the body of the patient.

In a non-limiting way, the source of energy can be:
a battery, preferably a battery with high energy density, for example a lithium battery,
a device for recovering mechanical energy, using for example the piezoelectric effect,
a biobattery capable of producing electricity by consuming chemical species, typically naturally present in the human or animal body, such as: glucose, carbohydrates, lipids, proteins,
a device for recovering solar energy, for example a photovoltaic module or a Grätzel cell,
a device for recovering thermal energy, for example a thermoelectric module using the Seebeck effect.

The source of energy is preferably capable of producing a voltage substantially less than or equal to 1.4V, in order to avoid the formation of Cl₂ from the ions of CI.

The device 2 can further comprise a voltage divider that allows to obtain a voltage for powering the electrolyzer substantially less than or equal to 1.4V. This is particularly useful when the source of energy 23 connected to the electrodes 21, 22 produces a voltage greater than 1.4V. The device 2 can comprise several pairs of cathodes 22 and of anodes 21. The source of energy 23 can be specific to each pair or shared.

A method for manufacturing the matrix 1 is now described according to one example. The method can comprise the manufacturing of the porous body 12, for example by 3D printing. The method can comprise the seeding of the pancreatic cells 13 in the porous body 12. After or before the seeding, the manufacturing method can comprise the encapsulation of the porous body 12 by the semipermeable wall 10. The method can comprise providing a matrix 1 devoid of pancreatic cells, and comprise the seeding in the matrix of the pancreatic cells 13, for example via the injection channel 14.

A method for delivering insulin is now described. The method can comprise the implantation of at least a part of the artificial pancreas device 2, and more particularly at least the matrix 1, in a human or animal body. This can in particular be carried out by an operation, adapted to the site of implantation and to the dimensions of the device. Preferably, the implantation is carried out at a limb or at the abdomen.

In the sense of the invention, animal can in particular mean large animals such as bovines, sports animals such as horses, pets such as dogs and cats, and laboratory animals such as rats, mice and monkeys.

The method can comprise the electric connection of the fluidic module and/or of the at least one anode and the at least one cathode to a source of electric energy, in particular when this source is remote. The method can also intend to control the closing and the opening of the electric circuit by means provided for this purpose.

According to one example, the method comprises a recharge of pancreatic cells, for example by injection of cells into the injection channel of the matrix. According to one example, the method comprises a suction of the pancreatic cells, before the recharge.

According to one example, the method comprises a recharge of the tank of the fluidic module, for example by injection.

The method can also intend to control the actuation of the fluidic module. According to one example, the method comprises the adjustment, by the regulator, of at least one parameter of the formation of the flow by the fluidic module. According to one example, the method comprises the actuation, and preferably the stopping, of the fluidic module of the device by the regulator. According to one example, the method comprises a modification by the regulator of the speed of the flow of liquid formed by the fluidic module of the device.

In light of the above description, it is clear that the invention proposes a solution, and in particular a matrix for receiving pancreatic cells and an artificial pancreas device allowing an improved delivery of insulin with respect to the existing solutions.

The invention is not limited to the embodiments described above and extends to all the embodiments covered by the invention. The present invention is not limited to the examples described above. Many other alternative embodiments are possible, for example by combining features described above, without going beyond the context of the invention. Moreover, the features described relative to one aspect of the invention can be combined with another aspect of the invention. For example, a method can comprise a step resulting from the implementation of a feature of the matrix and/or of the device, and vice versa.

### LIST OF THE NUMERICAL REFERENCES

1 Matrix for receiving pancreatic cells
10 Semipermeable wall
11 Inner volume
12 Porous body
120 First cavity set
1200 Cavity
121 Second cavity set
1210 Cavity
13 Pancreatic cell
14 Injection channel
140 Injection chamber
15 Fluid channel
2 Artificial pancreas device
20 Fluidic module
200 Electroactive coating
201 Tank
202 Pump
203 Regulator
204 Duct
205 Semipermeable membrane
21 Anode
22 Cathode
23 Source of electric energy
24 Source of electric energy
25 Solid support

1. A matrix for receiving pancreatic cells comprising:
a first set comprising at least one cavity comprising pancreatic cells,
a second set comprising at least one cavity devoid of pancreatic cells,
the first cavity set and the second cavity set not being fluidly connected to one another other than by the diffusion of a liquid through the walls of the porous body,
wherein the matrix comprises:
a semi-permeable wall at least partly defining an inner volume,
a porous body disposed in the inner volume, comprising:
the first set, and
the second set.

2. The matrix according to claim 1, wherein the first set and/or the second set comprises several cavities.

3. The matrix according to claim 2, wherein for each two consecutive cavities of the first set taken according to a first direction, a cavity of the second set is disposed between the two consecutive cavities of the first set.

4. The matrix according to claim 3, wherein for each two consecutive cavities of the first set taken according to a second direction, a cavity of the second set is disposed between the two consecutive cavities of the first set.

5. The matrix according to claim 1, wherein a distance between at least one cavity of the second set and at least one directly neighboring cavity of the first set taken from the center of the cavity of the first set to the center of the at least one cavity of the second set is between 0.5 mm and 3 cm.

6. The matrix according to claim 1, wherein the first set comprises a plurality of cavities interconnected and fluidly connected to an injection channel.

7. The matrix according to claim 1, wherein the second set comprises a plurality of interconnected cavities.

8. The matrix according to claim 1, wherein each cavity of the first set has a transverse cross-section having a smallest dimension substantially greater than the size of the pancreatic cells or of the islets of pancreatic cells.

9. The matrix according to claim 1, wherein the cavities of the first and of the second set extend according to a direction of principal extension in a manner parallel to each other, over a length substantially greater than or equal to 70% of the length of the porous body.

10. The matrix according to claim 1, wherein the porous body is chosen from the group consisting of chitosan, polyacrylamide, poly(p-phenyl-p-phthalamide), aramid nanofibers, polyvinyl alcohol.

11. The matrix according to claim 1, wherein the wall has a cutoff threshold substantially between 5.8 kDa and 8 kDa.

12. An artificial pancreas device intended to be implanted in the human or animal body and comprising the matrix for receiving pancreatic cells according to claim 1.

13. The device according to claim 12, further comprising a fluidic module configured to create a flow of liquid around and/or inside the matrix.

14. The device according to claim 13, wherein the fluidic module comprises a coating of the matrix, for example formed in the wall of the matrix, made from an electroactive polymer, the coating being capable of deforming the matrix.

15. The device according to claim 13, wherein the fluidic module is fluidly connected to the second cavity set.

16. The device according to claim 15, wherein the fluidic module comprises a tank and a pump, the pump being configured to induce a flow of liquid from the tank to inside the cavities of the second set.

17. The device according to claim 16, wherein the tank comprises at least one out of a nutrient for the pancreatic cells and an anti-inflammatory compound.

18. The device according to claim 15, wherein the fluidic module comprises a pump configured to induce a flow of liquid from a liquid outside of the device to inside the cavities of the second set.

19. The device according to claim 13, wherein the fluidic module comprises a regulator configured to regulate at least one parameter of the formation of the flow by the fluidic module.

20. The device according to claim 12, further comprising at least one anode and at least one cathode, a source of electric energy, the anode and the cathode being electrically connected to the source of electric energy, so that in the presence of bodily fluid, a closed electric circuit is formed so as to produce dihydrogen at the cathode and dioxygen at the anode, by electrolysis of the bodily fluid, at least one out of the cathode and the anode being disposed in the inner volume at least partly defined by the wall of the matrix.

21. The device according to claim 20, wherein at least one out of the cathode and the anode is disposed in the porous body of the matrix.

22. The device according to claim 20, wherein at least one out of the cathode and the anode is disposed on the porous body of the matrix.

23. A method for seeding the matrix for receiving pancreatic cells comprising:
providing a matrix capable of forming the reception matrix according to claim 1, comprising at least the porous body comprising:
a first cavity set intended to comprise pancreatic cells,
a second cavity set devoid of pancreatic cells,
the first cavity set and the second cavity set not being fluidly connected to one another,
a seeding of the first cavity set of the matrix by pancreatic cells.

24. A method for delivering insulin comprising the implantation of the artificial pancreas device according to claim 12, in a human or animal body.
